(19) United States
(12) Patent Application Publication
(10) Pub. No.: US 2022/0176569 A1
(43) Pub. Date: Jun. 9, 2022

(54) A ROBOTIC HAND

(71) Applicant: QBROBOTICS S.R.L., Cascina (Pisa) (IT)

(72) Inventors: Alberto BRANDO, PONTEDERA (Pisa) (IT); Riccardo PERSICHINI, MONTALTO DI CASTRO (Viterbo) (IT)

(21) Appl. No.: 17/602,670

(22) PCT Filed: Mar. 30, 2020
(86) PCT No.: PCT/IB2020/053013

§ 371 Date: Oct. 8, 2021

(30) Foreign Application Priority Data
Apr. 10, 2019 (IT) 102019000005558
Apr. 10, 2019 (IT) 102019000005562
Apr. 10, 2019 (IT) 102019000005564

(51) Int. Cl. B25J 15/00; A61F 2/58; B25J 9/10; B25J 15/02

(52) U.S. Cl. CPC B25J 15/0009; A61F 2/586; A61F 2002/704; B25J 15/0233; B25J 9/104

(57) ABSTRACT

A robotic hand is provided including a main body defining the body of the robotic hand and a longitudinal axis; a finger hinged to the main body; an attachment defining the attachment surface for attaching to an external member; and a hinge defining a mutual rotation axis between the attachment and main body so as to vary the angle between the longitudinal axis and the normal to the attachment surface.

This invention relates to a robotic hand of the type as recited in the preamble of the first claim.

As is well known, artificial hands and, to be precise, prosthetic hands are divided into two main classes: passive and active.

Passive prosthetic hands are used to replace a maimed body part in order to restore bodily integrity with particular attention to aesthetics. These hands are characterised by a stiff structure with non-motorised phalanges. They are not able to grip things. Active prosthetic hands have mechanical and/or electronic components that, by articulating the parts forming the hand between them, are able to reproduce various poses and therefore different grips.

The active artificial hands are, in turn, divided into two groups.

The first group is characterised by a number of actuators equal to the number of degrees of freedom and a control unit that controls the actuators independently from each other so that the hand is able to assume any pose/grip.

In view of the almost infinite number of poses that can be achieved, these artificial hands require an input that, needing to define each degree of freedom, is rich in information and particularly complicated to manage.

It should be pointed out that this drawback is evident in the case of prosthetic hands where the hand control is performed with contractions of the arm muscles and, therefore, the number of controls is extremely limited.

In addition, due to the large number of actuators, these artificial hands are very big and highly complex, both in terms of their construction and their programming. Therefore, the most widely used artificial hands are those that are under-implemented wherein the number of actuators is less than the number of degrees of freedom.

An example of an under-implemented artificial hand includes an actuator for each finger connected to the individual phalanges so as to control the rotation thereof. The fingers can, thus, be moved independently of each other.

In some cases, the under-implemented artificial hand may include a single actuator that, when connected to the phalanges, enables them to simultaneously rotate.

The above-mentioned prior art has some significant drawbacks.

In particular, the artificial hands, particularly evident in the under-implemented hands, have a limited number of poses and are, therefore, unable to take an object in any position.

Another drawback, therefore, lies in the fact that the above-mentioned drawbacks make it difficult for a known, under-implemented prosthetic hand to meet the patient's expectations or handling needs.

In this situation, the technical task underlying this invention is to devise a robotic hand able to substantially overcome at least some of the drawbacks mentioned.

Within the context of said technical task it is an important purpose of the invention to obtain a robotic hand capable of taking an object regardless of its position or shape.

The technical task and specified purposes are achieved by a robotic hand as claimed in the appended claim 1. Examples of preferred embodiments are described in the dependent claims.

The characteristics and advantages of the invention are clarified by the following detailed description of preferred embodiments thereof, with reference to the accompanying drawings, wherein:

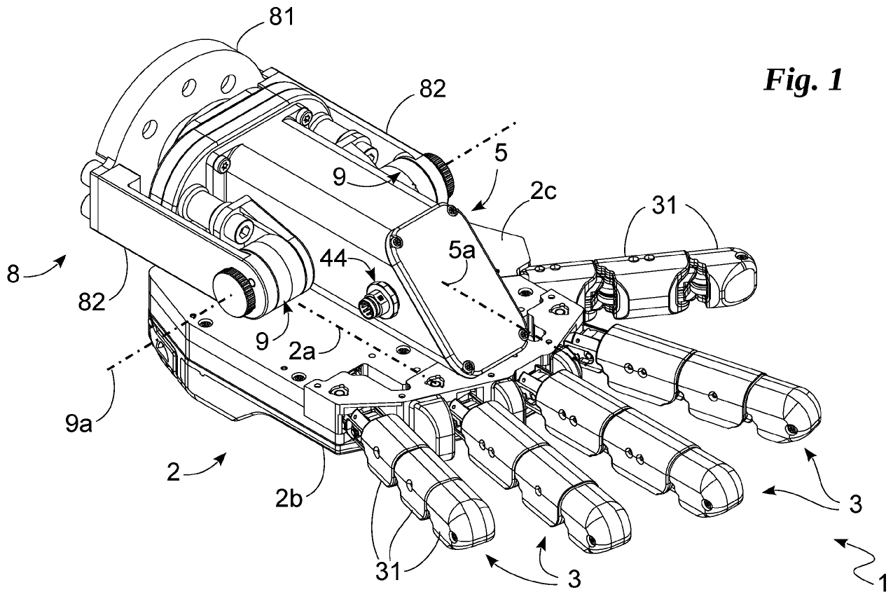

FIG. 1 shows, in scale, a robotic hand according to the invention;

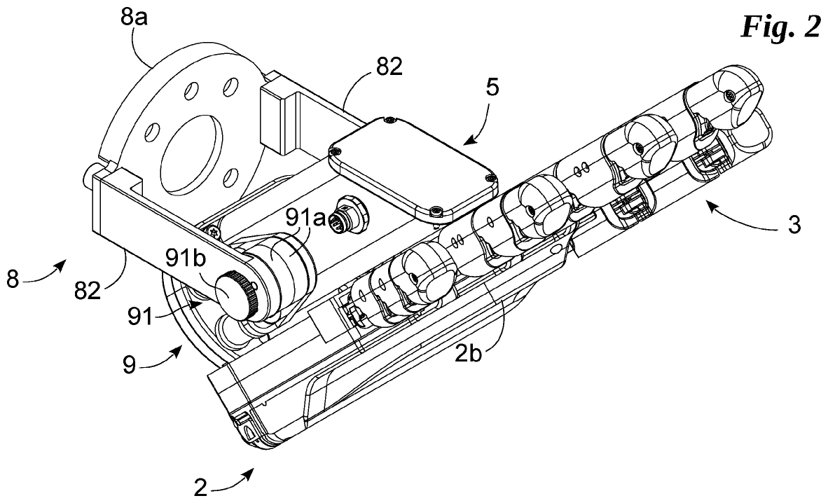

FIG. 2 illustrates, in scale, the robotic hand in FIG. 1 in a different position;

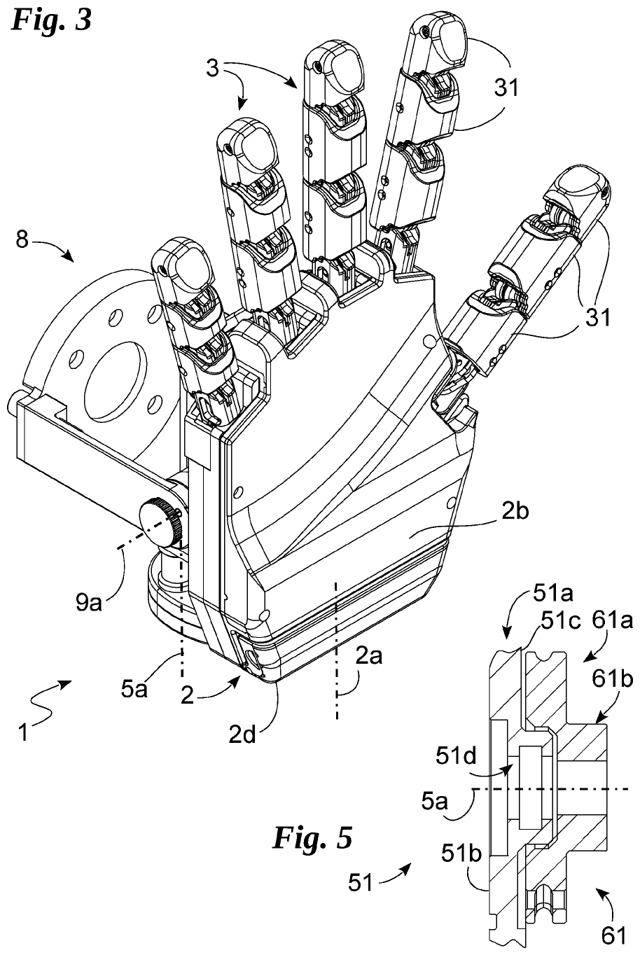

FIG. 3 presents, in scale, the robotic hand in an additional position;

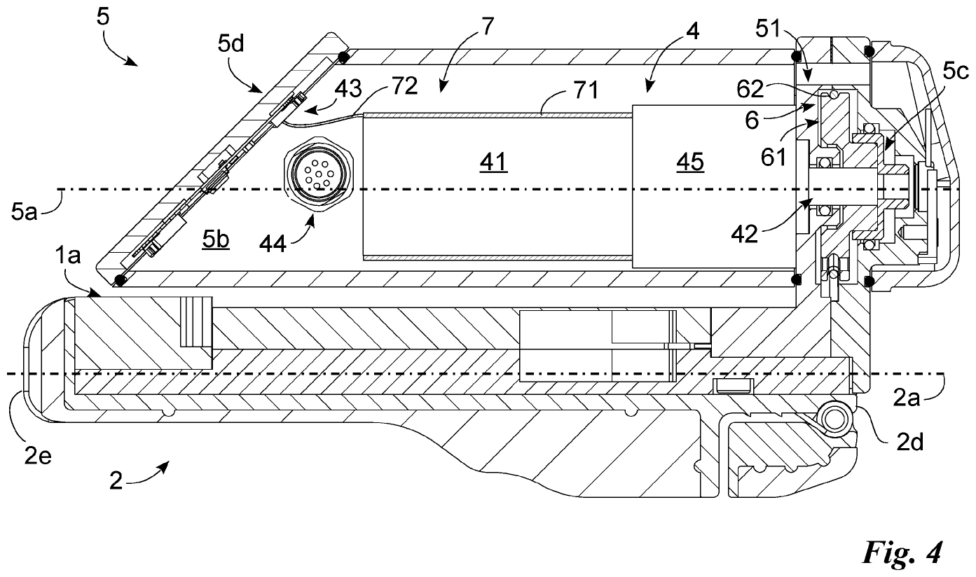

FIG. 4 shows, in scale, a cross-section of an assembly of the robotic hand according to the invention; and

FIG. 5 shows a detail in FIG. 4.

In this document, when measurements, values, shapes, and geometric references (such as perpendicularity and parallelism) are associated with words like “approximately” or other similar terms, such as “almost” or “substantially”, they shall be understood as except for errors of measurement or imprecisions due to errors of production and/or manufacturing and, above all, except for a slight divergence from the value, measurement, shape, or geometric reference with which it is associated. For example, if associated with a value, such terms preferably indicate a divergence of no more than 10% of the value itself.

Furthermore, when used, terms, such as “first”, “second”, “higher”, “lower”, “main” and “secondary” do not necessarily identify an order, relationship priority, or relative position, but they can simply be used to distinguish different components more clearly from one another.

The measurements and data provided in this text are to be considered as performed in ICAO International Standard Atmosphere (ISO 2533), unless otherwise indicated. Unless otherwise indicated, as evidenced by the discussions below, it should be understood that terms such as “processing”, “computer”, “computing”, “evaluation”, or the like, refer to the action and/or processes of a computer or similar electronic calculation device, which handles and/or processes data represented as physical, such as electronic sizes of logs of a computer system and/or their memories, other data similarly represented as physical quantities inside computer systems, logs or other information storage, transmission or display devices.

With reference to the figures, the reference number 1 denotes, as a whole, the robotic hand according to the invention.

The robotic hand 1 is designed to be fastened to an external member.

The external member is an external support bearing and suitably driving the hand 1. It is a robotic arm.

The hand 1 can preferably be a prosthetic hand designed to be used to replace a maimed body part in order to restore bodily integrity. In this case, the external member is a human limb.

The robotic hand 1 comprises a main body 2 defining the body of the robotic hand 1; and at least one finger 3 hinged to the main body 2.

The main body 2 defines a longitudinal axis 2a.

The main body 2 defines a palm 2b and a back 2c of the robotic hand 1.

The longitudinal axis 2a, palm 2b, and back 2c are basically parallel to each other.

The robotic hand 1 preferably comprises several fingers 3, preferably five.

Each finger 3 comprises at least one phalanx 31 and, in particular, at least two phalanges 31 and, to be precise, three phalanges 31.

The robotic hand 1 comprises a motor unit 4 to control movement in relation to the main body 2 of at least one finger 3 and, in particular, of at least one phalanx 31;

and, preferably, a motion control kinematic system of one or more fingers 3 depending on the motor unit 4.

The motor unit 4 comprises at least one motor 41; one output shaft 42 for the motion from the motor 41; preferably a board 43 to control the motor 41; and, suitably, at least one connector 44 (wireless or wired) designed to enable the control of the motor unit 4 and, therefore, of the robotic hand 1.

The motor unit 4 may comprise a gear unit 45 between the motor 41 and the shaft 42 for each motor 41.

The motor 41 is electric.

The board 43 can be used to enable an external unit to control the motor 41 and, thus, the robotic hand 1.

The robotic hand 1 is preferably under-implemented and, as a result, comprises a number of motors 41 that is lower than the degrees of freedom of the hand 1. The robotic hand 1 preferably comprises two motors 41 at the most and, specifically, only one motor 41.

The kinematic control system is designed to move one or more phalanges 31 enabling the robotic hand 1 to assume one or more poses.

It is fastened directly to the main body 2 and, in particular, housed in a housing compartment of the main body 2.

The kinematic control system may be of a known type. One non-exhaustive example of a kinematic control system is described in WO2017199127 from pg. 4, I. 4, to pg. 17, I. 5, and in FIGS. 1a-6c (these parts of document WO2017199127 are included here by way of reference). The kinematic control system can thus comprise a control cable for one or more fingers 3.

The motor unit 4 is firmly fastened to the main body 2, suitably at the back 2c.

The robotic hand 1 can preferably comprise a containment chamber 5 for the motor unit 4 to fasten the motor unit 4 to the main body 2 overhanging at the back 2c so that a free gap 1a is defined between the chamber 5 and the main body 2 (specifically, the control kinematic system).

The chamber 5 is fastened overhanging the main body 2 at the back 2c and preferably almost entirely overlapping at the wrist. As a result, the robotic hand 1 has said free gap 1a between the chamber 5 and the main body 2 (specifically, the kinematic control system—highlighted in FIG. 4).

The thickness of the free gap 1a (calculated perpendicularly to the wrist 2c) is at least 1 mm and, specifically, basically between 2 mm and 5 mm so as to enable the robotic hand 1 to cover the whole main body 2 and one or more fingers 3. For example, the hand 1, if there are five fingers 3, can wear a normal glove.

The robotic hand may comprise a transmission 6 of the motion from the motor unit 4 located in the chamber 5 to the kinematic control system fastened to the main body 2 and suitably located in said housing compartment.

The chamber 5 defines a main extension axis 5a.

The chamber 5 is almost parallel to the main body 2. As a result, the main extension axis 5a may be basically parallel to the longitudinal axis 2a.

It comprises a partition 51 dividing the same chamber 5 into a first sub-chamber 5b and a second sub-chamber 5c.

The chamber 5 is fastened to the main body 2 by the partition 51.

The first sub-chamber 5b is preferably watertight.

The first sub-chamber 5b may be divided by the partition 51, along the main extension axis 5a, into a first sub-chamber and a second sub-chamber placed in fluidic connection through an ring-shaped section partially surrounding the second sub-chamber 5c.

The first sub-chamber 5b houses the motor 41; gear unit 45, if any; and, suitably, the board 43. As a result, all the electronic/electrical components of the robotic hand 1 are housed in the first sub-chamber 5b.

The second sub-chamber 5c houses the transmission 6.

The motor 41 transmits the motion to the transmission 6 thanks to the shaft 42 through the partition 51. As a result, the partition 51 defines an engagement hole on the shaft 42 designed to enable the motor 41, located in the first sub-chamber 5b, to control the transmission 6, located in the second sub-chamber 5c; and, suitably, bearings, bushings, or other similar means designed to enable the shaft 42 to rotate in relation to the partition 51.

The partition 51 is designed to bear the motor 41 and the shaft 42 and comprises a wall 51a designed to support the motor 41, shaft 42, and gear unit 45, if any.

The wall 51a (FIG. 5) defines a first support surface 51b for the motor 41 (to be precise, for the gear unit 45) and a second surface 51c opposite said first surface 51b.

The first surface 51b may be basically flat.

The second surface 51c may have a basically cap-shaped profile so as to define, at the through hole, a thickening 51d (suitably along the main extension axis 5a) designed to enable the partition 51 to bear the shaft 42 and, thus, the motor 41. The thickening 51d houses a seal enabling the shaft 42 to rotate.

The board 43 is fastened to the chamber 5 and, in particular, to a base panel 5d of the chamber 5 and, preferably, the first sub-chamber 5b. Said base panel 5d is transverse to the main extension axis 5a so as to have a wider anchoring surface (compared to having a panel 5d perpendicular to the axis 5a) for the board 43 and to define a less angular shape for the chamber 5.

The transmission 6 comprises at least one pulley 61, specifically only one, designed to be rotated by the motor unit 4; a drive cable 62 designed to be moved by the pulley controlling the control cable, and, thus, the kinematic control system; and, suitably, an encoder of the angular position of the pulley 61.

The pulley 61 may be the final element of the transmission 6.

The encoder is located in the first sub-chamber 6b.

The drive cable 62 may be the control cable and, thus, the pulley 61 directly controls the control cable. The transmission 6 may comprise one or more return elements (pulleys) designed to enable the drive cable 62 to control the control cable.

Alternatively, the pulley 61 indirectly controls the control cable. The transmission 6 may comprise one or more return elements (pulleys) designed to enable the drive cable 62 to control the control cable.

The chamber 5 may comprise a through opening for the drive cable 62, through which the drive cable 62 passes from the chamber 5 (specifically, the second sub-chamber 5c) to the kinematic control system, suitably located in the housing compartment.

The pulley 61 (FIG. 5) may have a profile that is basically counter-shaped to the second surface 51c and, in particular, to the hat. It comprises a first ring 61a defining at least one sliding groove for the drive cable 62 and a second engagement ring 61b for engaging the shaft 42; and a connection firmly fastening the rings 61a and 61b together.

The first ring 61a is at least partially and, specifically, almost totally offset from the second ring 61b along the main extension axis 5a. It is closer to the motor, along the main extension axis 5a, than the second ring 61b.

The first ring 61a is designed to overlap the thickening 51d normally to the main extension axis 5a. It is, thus, designed to rotate around said thickening 51d.

The second ring 61b is designed to overlap the thickening 51d along the main extension axis 5a.

It should be highlighted how this pulley 61 profile makes it possible both to reduce the bending moment on the motor shaft and/or to arrange a sealing ring on the shaft without increasing the overall dimensions of the transmission 6 and making it possible to form a watertight chamber 5b.

The robotic hand 1 may comprise a magnetic field absorber 7.

The absorber 7 is designed to absorb the magnetic fields produced by at least the motor 41, in particular, by the motor unit 4 and, preferably, coming from outside.

The absorber 7 is housed in the chamber 5 and, in particular, in the first sub-chamber 5b in order to avoid the magnetic fields spreading outside of it.

The absorber 7 may comprise a Faraday cage 71 surrounding and, specifically, wrapping around at least part of the motor 41 and absorbing magnetic fields by transforming them by induction into electric current; and a discharge apparatus 72 for said electric current.

The Faraday cage 71 may surround and, specifically, wrap around at least 50% and, more specifically 75%, and, even more specifically, the whole of the motor 41.

The Faraday cage 71 may comprise a wrap-around net, specifically, in contact, preferably direct contact, with the motor 41, suitably the gear unit 45, and/or any encoder connection cables.

Alternatively or additionally, the Faraday cage 71 may comprise a conductive paint sprayed on at least the inner surfaces of the chamber 5 and, to be precise, of the first sub-chamber 5b.

In some cases, the Faraday cage 71 may comprise a net wrapping around the motor 41, said conductive paint sprayed on at least the inner surfaces of the chamber 5, and, to be precise, of the first sub-chamber 5b.

The discharge apparatus 72 may be designed to discharge the electrical current collected from the cage 71 to the outside. Alternatively or additionally, it may use said current to power the robotic hand 1.

The discharge apparatus 72 may comprise an electrical cable that can be connected to an external device/network capable of absorbing said current produced by the Faraday cage 71.

The robotic hand 1 comprises an attachment 8 defining an attachment surface 8a to the above-mentioned external member; and at least one hinge 9 defining a mutual rotation axis 9a between the attachment 8 and the main body 2 and, thus, between the attachment 8 and the rest of the robotic hand 1 so as to vary the angle between the longitudinal axis 2a and the normal to the attachment surface 8a.

The rotation axis 9a is almost transverse and, in particular, basically perpendicular to the longitudinal axis 2a. Specifically, it is skew in relation to the longitudinal axis 2a.

The main body 2 defines a face 2d proximal to the attachment 8 and a face 2e distal to the attachment 8.

The rotation axis 9a is between the proximal face 2d and the distal face 2e and, in particular, placed opposite the palm 2b in relation to the back 2c.

The normal to the attachment surface 8a is basically perpendicular to the rotation axis 9a.

The attachment 8 comprises a plate 81 for fastening the main body 2 to the external member; and at least one arm 82 fastening the hinge 9 overhanging the plate 81.

The plate 81 may be ring-shaped.

The arm 82 is designed to place the rotation axis 9a between the proximal face 2d and the distal face 2e. In particular, the arm 82 defines a distance between the attachment surface 8a and the rotation axis 9a basically ranging between 1 cm and 15 cm, specifically between 5 cm and 10 cm, and more specifically between 6 cm and 7 cm.

The attachment 8 preferably comprises two arms 82 enclosing the motor 41, and in particular the chamber 5, between them. The robotic hand 1 comprises two hinges 9 each of which are designed to fit between the arm 82 and the motor 41 and, specifically, between the arm 82 and the chamber 5.

The distance, along the rotation axis 9a, between the arms 82 and, in particular, between the hinges 9 is at least equal to that of the chamber 5.

The hinge 9 defines a variation range for the angle between the longitudinal axis 2a and normal to the attachment surface 8a basically ranging between 0° and 45°, and specifically between 0° and 90°, in at least one rotation direction, and, in some cases, in both rotation directions (in this case the distance, along the rotation axis 9a, between the arms 82 is at least equal to that of the main body 2).

Each hinge 9 comprises a locking system 91 of the mutual rotation between the attachment 8 and the main body 2.

The locking system 91 defines a plurality of locking positions for mutual rotation between the attachment 8 and the main body 2 and, thus, stable hand configurations with different angles between the longitudinal axis 2a and the attachment surface 8a.

The longitudinal axis 2a and the attachment surface 8a in the locking positions, i.e. in stable configurations, are equally angularly spaced apart.

In particular, the angle between the longitudinal axis 2a and the attachment surface 8a varies between the locking positions and, in particular, varies by an angle substantially less than 30°, specifically less than 15°, and preferably basically ranging between 10° and 5°, and more preferably basically 7.5°.

The locking system 91 comprises two gears 91a with front teeth designed to mutually engage by locking the rotation; and control means 91b for the mutual rotation between the gears 91a.

One gear 91a is integral with the main body 2 and the other wheel 91a is integral with the attachment 8.

The control means 91b are designed to selectively enable or prevent mutual rotation of the gears 91a. They enable both a distancing between the gears 91a along the rotation axis 9a that, by disengaging the teeth, enables the gears 91a to mutually rotate, and a bringing closer of the gears 91a along the rotation axis 9a that, by engaging the teeth, prevents the gears 91a from mutually rotating.

The control means 91b may comprise a threaded pin engaged to one of the gears 91a and designed to rotate in relation to it by controlling its movement along the rotation axis 9a.

The control means 91b can be automatic and, thus, include their own actuator. Alternatively, they are manual and, therefore, include a control grip surface for the means 91b for the operator.

Finally, it should be highlighted how the rotation given by the hinge 9 can be manual (performed, for example, by means of the locking system 91 as described above). Alternatively, it can be automatic. This rotation may be controlled by the locking system 91 as described above or, alternatively or additionally, the robotic hand 1 may comprise a drive (e.g. an electric motor) for the rotation of at least the main body 2.

The operation of the robotic hand 1 described above in structural terms, is as follows.

The robotic hand 1 is fastened to an external member, thanks to the attachment 8, at the attachment surface 8a and, thus, data connected to an external control unit of the robotic hand 1 via the connector 44.

The robotic hand 1 is, thus, ready to be used.

The external unit controls the gripping of an object based on an external signal from an operator.

In response to this command the hinge 9 activates\enables the rotation of at least the main body 2 and one or more fingers 3 in relation to the attachment 8 and, thus, to the external member until the desired position, and in particular the closest locking position to said desired position, is reached. At this point, the board 43, based on said external signal, controls the actuation of the motor 41 that, through the gear unit 45, rotates the shaft 42 and, thus, the pulley 61. The pulley 61 pulls the drive cable 62 that, by controlling the control cable, brings the fingers 3 into the desired gripping position.

The robotic hand 1 according to the invention achieves important advantages.

In fact, the innovative introduction of the hinge 9, enabling a mutual rotation between the attachment 8 (i.e. the external member) and the rest of the robotic hand 1, instils the same hand with an extraordinary flexibility of use and, thus, the gripping of objects in any position.

It should be specified that these advantages are guaranteed by the special positioning of the rotation axis 9a, which enables an extraordinary range of rotation. Another advantage is the division of the chamber 5 into sub-chambers 5b and 5c, and the special arrangement of the various electrical and mechanical components that eliminates the risk of interference between them. This aspect is enhanced by the presence of the absorber 7 that, by absorbing all the magnetic fields produced by the electrical components of the robotic hand 1, ensures there is no interference even with other devices.

Another important advantage is the definition of the free gap 1a that enables the robotic hand 1 to wear a normal glove.

Variations may be made to the invention that fall within the scope of the inventive concept defined in the claims.

For example, the robotic hand 1 may comprise an additional hinge defining an additional rotation axis between the attachment 8 and the main body 2.

The additional rotation axis is basically transverse and, specifically, basically perpendicular to the rotation axis 9a.

The additional rotation axis is, suitably, basically parallel to the attachment surface 8a.

Said additional hinge may be similar to the hinge 9.

In this context, all the components may be replaced with equivalent elements and the materials, shapes, and dimensions may be as desired.

1. A robotic hand comprising:
a main body defining the body of said robotic hand and a longitudinal axis;
at least one finger hinged to said main body;
an attachment defining an attachment surface for attaching to a member external to said main body and therefore to said robotic hand;
and
a hinge defining a mutual rotation axis between said attachment and said main body so as to vary the angle between said longitudinal axis and the normal to said attachment surface.

2. The robotic hand according to claim 1, wherein said main body defines a proximal face proximal to said attachment, and a distal face distal to said attachment; and wherein said rotation axis is between said proximal face and said distal face.

3. The robotic hand according to claim 2, wherein said rotation axis is basically perpendicular to said normal to said attachment surface and to said longitudinal axis.

4. The robotic hand according to claim 3, wherein said attachment comprises a fastening plate that fastens said main body to said external member; and at least one arm fastening said hinge overhanging said fastening plate.

5. The robotic hand according to claim 1, wherein said hinge comprising a locking system for locking the mutual rotation between said attachment and said main body; and
wherein said locking system defines a plurality of locking positions for locking said mutual rotation between said attachment and said main body; and wherein said locking positions are equally angularly spaced apart from one another.

6. A robotic hand comprising:
a main body defining the body of said robotic hand and a longitudinal axis;
at least one finger hinged to said main body;
a motor unit;
a motion control kinematic system of said at least one finger depending on said motor unit;
said motor unit comprising at least one motor designed to control said control kinematic system;
and
an absorber of magnetic fields produced at least by said motor.

7. The robotic hand according to claim 6, comprising a containment chamber of said motor unit and of said absorber fastened to said main body.

8. The robotic hand according to claim 7, wherein said control kinematic system is external to said chamber; and wherein said robotic hand comprises a transmission of the motion from said motor unit in said chamber to said control kinematic system external to said chamber.

9. The robotic hand according to claim 8, wherein said chamber comprises a partition dividing said chamber into a first sub-chamber housing said motor and a second sub-chamber housing said transmission; and wherein said motor unit comprises a shaft driven by said motor in said first sub-chamber and controlling said transmission in said second sub-chamber.

10. The robotic hand according to claim 6, wherein said absorber comprises a Faraday cage winding at least said motor and designed to absorb said magnetic fields by transforming them by induction into electric current and a discharge apparatus of said electric current.

11. A robotic hand comprising:
a main body defining the body of said robotic hand and a longitudinal axis;
at least one finger hinged to said main body;
a motor unit;
a motion control kinematic system of said at least one finger depending on said motor unit fastened to said main body;
said motor unit comprising at least one motor designed to control said control kinematic system;
a containment chamber of said motor unit defining a main extension axis; and
a transmission of the motion from said motor unit in said chamber to said external control kinematic system to said chamber.

12. The robotic hand according to claim 11, wherein said chamber is fastened overhanging said main body so that said robotic hand defines a free gap between said main body and said chamber.

13. The robotic hand according to claim 11, wherein said motor unit comprises at least one motor, a shaft transmitting the motion from said motor to said transmission and a control board of said motor; wherein said chamber comprises a partition dividing said chamber into a first sub-chamber and a second sub-chamber; wherein said first sub-chamber houses said motor and said board; wherein said second sub-chamber houses said transmission.

14. The robotic hand according to claim 11, wherein said transmission comprises a pulley designed to be rotated by said motor unit and a drive cable designed to be driven by said pulley controlling said control kinematic system; and wherein said pulley comprises a first ring defining a sliding groove for said drive cable and a second ring binding to said shaft; and a connection firmly fastening said rings (61a, 61b) to each other.

15. The robotic hand according to claim 14, wherein said first ring is at least partially offset in relation to said second ring along said main extension axis.

16. The robotic hand according to claim 14, wherein said first ring is at least partially offset in relation to said second ring along said main extension axis and is closer to said motor than said second ring.
